(19) United States
(12) Patent Application Publication
(10) Pub. No.: US 2026/0199270 A1
(43) Pub. Date: Jul. 16, 2026

(54) METHODS FOR TREATING NEURODEGENERATIVE DISORDERS WITH TRAMIPROSATE

(71) Applicant: Alzheon, Inc., Framingham, MA (US)

(72) Inventors: Susan Abushakra, Framingham, MA (US); John Hey, Framingham, MA (US); Martin Tolar, Framingham, MA (US)

(21) Appl. No.: 19/135,046

(22) PCT Filed: Dec. 4, 2023
(86) PCT No.: PCT/US2023/082352

§ 371 Date: Jun. 2, 2025

Related U.S. Application Data
(60) Provisional application No. 63/429,637, filed on Dec. 2, 2022.

(51) Int. Cl. A61K 31/185; A61K 31/19; A61K 38/16; A61K 39/395; A61P 35/00

(52) U.S. Cl. CPC A61K 31/185; A61K 31/19; A61K 38/164; A61K 39/3955; A61P 35/00

(57) ABSTRACT

Provided herein are methods for treating Alzheimer's disease using a combination of tramiprosate, a tramiprosate prodrug or an active tramiprosate metabolite with at least one amyloid plaque clearing agent.

### RELATED APPLICATIONS

This application claims priority to U.S. provisional application No. 63/429,637, filed Dec. 2, 2022, the entire contents of which are incorporated herein by reference.

### BACKGROUND

Alzheimer's disease (AD) is a progressive, neurodegenerative disorder affecting over 6 million people in the United States. AD is currently ranked as the sixth leading cause of death and is characterized by extracellular amyloid-β (Aβ) plaques generated by the deposition of beta-amyloid protein and intracellular buildup of tau proteins, resulting in intracytoplasmic neurofibrillary tangles (NFT). Aβ causes synaptic impairment and neurodegeneration, consequently contributing to the cognitive dysfunction and progressive cognitive and functional deficits observed in AD. Recently, immunotherapies have emerged as a potential strategy to actively clear aggregated Aβ (amyloid plaques) and reduce the accumulation of Aβ in the brain and potentially treat the underlying cause of AD. Such therapies include both active and passive immunization.

Although certain immunotherapies have been shown to reduce Aβ plaques in the brain, they come with significant risk. Immunotherapies, such as the administration of certain monoclonal antibodies, break down Aβ aggregated plaques from brain parenchyma and the vessel wall by forming immune complexes, recruiting microglia, and inducing inflammation. These immune complexes and the inflammatory responses they induce injure cerebral vessel walls leading to amyloid-related imaging abnormalities including vasogenic edema (ARIA-E) and cerebral hemorrhage (ARIA-H). These ARIA events can manifest as headaches, worsening confusion, dizziness, visual disturbances, nausea, and seizures. ARIA-E and ARIA-H can lead to serious events like stroke, seizures, and status epilepticus, and even be fatal. Moreover, the risk of ARIA events is especially high for subjects who are taking an anticoagulant therapeutic and even more so if the subject also has evidence of cerebral amyloid angiopathy (“CAA”). It has recently been shown that AD subjects being treated with both an anticoagulant and an amyloid plaque clearing antibody have higher risk of vascular injury or amyloid-related imaging abnormalities than AD subjects who are treated with only the antibody. That risk is even greater if the subject treated with the anticoagulant has evidence of CAA.

Safe and effective alternative strategies for the use of immunotherapies in the context of treating AD are therefore needed.

### SUMMARY

ALZ-801, a promising new treatment for AD, is currently being investigated in clinical trials for subjects with early AD (MMSE>22). ALZ-801 reduces the brain burden of soluble aggregated forms of Aβ42 (called oligomers), the building block and main toxic components of plaques, by clearing Aβ42 from brain to plasma. See e.g., FIG. 2. Therefore, unlike recent antibody-based immunotherapies which target and bind plaque directly, ALZ-801 blocks formation of oligomers to prevent new plaque formation. In addition, ALZ-801 reduces the burden of Aβ40, the main component of vascular amyloid, by clearing it from brain to plasma, and also induces early reduction in plasma p-tau, which is a marker of Aβ-induced neuronal stress and injury. See e.g., FIG. 1 and FIG. 3.

The significant reduction of Aβ40 suggests that ALZ-801 decreases the burden of Aβ40 in blood vessels, leading to healthier small-to-medium sized cerebral blood vessels. This is further supported by the early significant decrease in the neuronal injury marker plasma p-tau, which indicates decreased amyloid-induced neuronal stress. See e.g., FIG. 3. Unlike the amyloid immunotherapies, AD patients treated with ALZ-801 for 1 year showed no events of amyloid-related imaging abnormalities with edema (ARIA-E) and no macro-hemorrhages (bleeds larger than 1 cm in diameter).

Provided herein are administration protocols for treating a subject suffering from AD using a combination of ALZ-801 and at least one amyloid plaque clearing agent (e.g., an antibody). The disclosed protocols first comprise the administration of ALZ-801 for a first period of time in an effort to counteract and reduce Aβ42 toxicity, and to lower vascular amyloid burden. Next, because amyloid plagues may act as reservoirs of toxic oligomers and since ALZ-801 does not directly target pre-existing plaques, an amyloid plaque clearing agent is then introduced as part of a concurrent treatment with ALZ-801 for a second period of time. In one aspect, it is postulated that clearance of Aβ40 resulting from pre-treating subjects with ALZ-801 for the first period of time leads to an improvement in the overall health and function of cortical small and medium vessels that typically accumulate amyloid. Aβ40 is the main component of vascular amyloid which deposits in and around the smooth muscle layer and reduces vascular elasticity and integrity. Thus, it is believed that if pre-treated with ALZ-801 for a period of time, a subject's risk of experiencing vascular injury or amyloid-related imaging abnormalities such as ARIA-E or ARIA-H are reduced when taking the amyloid plaque clearing agent. This reduction in risk would be particularly advantageous for subjects taking anticoagulants, especially those who also have evidence of CAA. Finally, after the second period of time, the amyloid plaque clearing agent is no longer administered and treatment with ALZ-801 continues thereafter as maintenance therapy to prevent the re-accumulation of Aβ plaques in AD brain.

Other compounds which act in a similar manner to reduce aggregated forms of Aβ42 like ALZ-801 are also contemplated to be useful in the present administration protocols. These include, but are not limited, tramiprosate, tramiprosate prodrugs other than ALZ-801, and active metabolites of tramiprosate.

### BRIEF DESCRIPTION OF THE FIGURES

FIG. 1 illustrates the effect on plasma Abeta40 (change from baseline) following a 52 week treatment with 265 mg ALZ-801 administered orally twice daily to subjects who are APOE4+ and have early AD.

FIG. 2 illustrates the effect on plasma Abeta42 (change from baseline) following a 52 week treatment with 265 mg ALZ-801 administered orally twice daily to subjects who are APOE4+ and have early AD.

FIG. 3 illustrates the effect on plasma p-tau₁₈₁ (change from baseline) following a 52 week treatment with 265 mg ALZ-801 administered orally twice daily to subjects who are APOE4+ and have early AD.

### DETAILED DESCRIPTION

As part of a first embodiment, provided herein is a method of treating a subject suffering from Alzheimer's disease comprising the steps of:
a. administering to the subject an effective dose of tramiprosate, a tramiprosate prodrug, a tramiprosate metabolite, or a deuterated form of any of the foregoing for a first period of time;
b. co-administering to the subject for a second period of time:
   i. an effective dose of tramiprosate, a tramiprosate prodrug, a tramiprosate metabolite, or a deuterated form of any of the foregoing; and
   ii. an effective dose of an amyloid plaque clearing agent; and
c. administering to the subject an effective dose of tramiprosate, a tramiprosate prodrug, a tramiprosate metabolite or a deuterated form of any of the foregoing thereafter.

As part of a second embodiment, provided herein is a method of treating a subject suffering from Alzheimer's disease comprising the steps of:
a. administering to the subject an effective dose of ALZ-801 or a deuterated form thereof for a first period of time;
b. co-administering to the subject for a second period of time:
   i. an effective dose of ALZ-801 or a deuterated form thereof; and
   ii. an effective dose of an amyloid plaque clearing agent; and
c. administering to the subject an effective dose of ALZ-801 or a deuterated form thereof thereafter.

As part of a third embodiment, provided herein is a method of treating a subject suffering from Alzheimer's disease comprising the steps of:
a. administering to the subject an effective dose of tramiprosate or a deuterated form thereof for a first period of time;
b. co-administering to the subject for a second period of time:
   i. an effective dose of tramiprosate or a deuterated form thereof; and
   ii. an effective dose of an amyloid plaque clearing agent; and
c. administering to the subject an effective dose of tramiprosate thereafter.

As part of a fourth embodiment, provided herein is a method of treating a subject suffering from Alzheimer's disease comprising the steps of:
a. administering to the subject an effective dose of 3-SPA for a first period of time;
b. co-administering to the subject for a second period of time:
   i. an effective dose of 3-SPA; and
   ii. an effective dose of an amyloid plaque clearing agent; and
c. administering to the subject an effective dose of 3-SPA thereafter.

“Tramiprosate” (homotaurine, 3-amino-1-propanesulfonic acid (3-APS), or Alzhemed™) is an oral amyloid anti-aggregation agent which inhibits formation of amyloid beta oligomers and reduces oligomer-related neurotoxicity in the brain. See e.g., J Nutr Health Aging 13, 550-557 (2009). Tramiprosate has the following chemical structure:

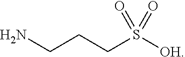

A “tramiprosate prodrug” or “prodrug of tramiprosate” refers to a chemical compound that, after administration to a subject, is metabolized into tramiprosate. Such prodrugs include, but art not limited to, those having the formula:

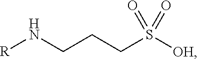

and pharmaceutically acceptable salts, wherein R is (AA¹)_{q}(AA²)ₜ-H; AA¹ and AA² are each independently selected from alanine (Ala), cysteine (Cys), aspartic acid (Asp), glutamic acid (Glu), phenylalanine (Phe), glycine (Gly), histidine (His), isoleucine (Ile), lysine (Lys), leucine (Leu), methionine (Met), asparagine (Asn), proline (Pro), glutamine (Gln), arginine (Arg), serine (Ser), threonine (Thr), valine (Val), tryptophan (Trp), tyrosine (Tyr), β-alanine (β-ALA), and γ-aminobutyric acid (GABA); q is 1; and t is 0 or 1. In certain aspects, the tramiprosate prodrug is ALZ-801. Additional tramiprosate prodrugs useful in the present invention are disclosed in WO 2015/143447, WO 2009/019534, WO 2017/027582, WO 2004/113275, WO 2006/085149, WO 1994/022437, WO 2000/064420, WO 1999/040909, WO 1999/059571, WO 2004/112762, and WO 2018/156845, the contents of each of which are incorporated herein by reference.

ALZ-801 refers to valyl-3-amino-1-propanesulfonic acid, a prodrug of tramiprosate, represented by the structure below:

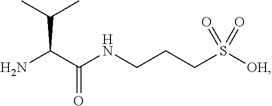

An “active tramiprosate metabolite” refers to a metabolized form of tramiprosate which continues to produce effects in the body. Active tramiprosate metabolites include the compound 3-SPA, having the formula

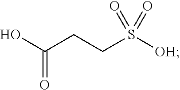

and pharmaceutically acceptable salts thereof. See e.g., WO 2020/028348, the entire contents of which are incorporated herein by reference.

Deuterated forms of tramiprosate or a tramiprosate prodrug, such as ALZ-801, are described in United States patent publication Nos. US2009/0076167 and US2018/0273471.

“Effective amount” or “effective dose” refers to an amount of a compound or biologic described herein that is sufficient to achieve the desired therapeutic effect (such as a reduction in plaque, a reduction in one or more fluid or imaging biomarkers related to AD, or treating AD) under the conditions of administration. Effective amounts can vary, as recognized by one of ordinary skill in the art, depending on e.g., the severity of the AD, the route of administration, the sex, age and general health condition of the patient, excipient usage, the possibility of co-usage with other therapeutic treatments such as use of other agents and the judgment of the treating physician or other medical provider. In some aspects, an effective amount of the amyloid plaque clearing agent is an amount sufficient to reduce plaque (e.g., as measured by PET scans, CSF or plasma amyloid biomarker, or other means known to those skilled in the art). In some aspects, an effective amount of tramiprosate, a tramiprosate prodrug (e.g., ALZ-801) or an active tramiprosate metabolite (e.g., 3-SPA) is an amount sufficient to reduce one or more biomarkers related to AD (e.g., plasma levels of Aβ40, Aβ42, and/or p-tau₁₈₁, or MRI measures such as hippocampal volume) below a certain threshold. In some aspects, an effective amount of a compound or biologic described herein refers to a dosage of between 0.01-100 mg/kg body weight/day.

As used herein, the term “treat”, “treating” or “treatment” means reversing, alleviating, inhibiting, or slowing the progress of Alzheimer's disease (AD), including cognitive decline or one or more symptoms associated therewith.

The term “co-administering” as used herein with respect to the administration of tramiprosate, a tramiprosate prodrug, or an active tramiprosate metabolite with at least one amyloid plaque clearing agent means that the at least one amyloid plaque clearing agent may be administered prior to, contemporaneously with, or after the administration of tramiprosate, a tramiprosate prodrug, or an active tramiprosate metabolite. Thus, it is not necessary that tramiprosate, a tramiprosate prodrug, or an active tramiprosate metabolite be administered at the same time with at least one amyloid plaque clearing agent. In one aspect, however, effective amounts of tramiprosate, a tramiprosate prodrug, or an active tramiprosate metabolite and at least one amyloid plaque clearing agent will be present in the subject at the same time. Similarly, in certain aspects, co-administration will refer to the fact that tramiprosate, a tramiprosate prodrug, or an active tramiprosate metabolite are administered at significantly different times than the at least one amyloid plaque clearing agent, but the effects of the at least one amyloid plaque clearing agent with tramiprosate, a tramiprosate prodrug, or an active tramiprosate metabolite are present at the same time. As used herein, a sub-therapeutic dose refers to a dosage amount of an amyloid plaque clearing agent that is less than the amount required to reverse, alleviate, inhibit, or slowing the progress of Alzheimer's disease (AD), including cognitive decline, when used as a monotherapy, i.e., in the absence of co-administering tramiprosate, a tramiprosate prodrug, or an active tramiprosate metabolite. In certain aspects, a sub-therapeutic dose of an amyloid plaque clearing agent is an amount that is sufficient to reduce amyloid plaque burden.

The term “subject” and “patient” are used interchangeably. In one aspect, the subject is a human. In some aspects, the subject is a human aged 100 years old or less, 95 years old or less, 90 years old or less, or 85 years old or less. In other aspects, the subject is a human aged 65-100 years old, 65-95 years old, 65-90 years old, or 65-85 years old. In yet other aspects, the subject is human age 58 years old or older. In still other aspects the human is in need of treatment.

Suitable routes for administering tramiprosate, a prodrug of tramiprosate (e.g., ALZ-801), an active metabolite of tramiprosate (e.g., 3-SPA), and an amyloid plaque clearing agent include, but are not limited to, orally, parenterally, by inhalation spray, topically, nasally, buccally, or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intraperitoneally or intravenously. Sterile injectable forms of the pharmaceutical compositions described herein may be aqueous or oleaginous suspension. These suspensions may be formulated according to techniques known in the art using suitable dispersing or wetting agents and suspending agents. In some aspects, tramiprosate, a prodrug of tramiprosate (e.g., ALZ-801), an active metabolite of tramiprosate (e.g., 3-SPA), or an amyloid plaque clearing agent are administered orally. In some aspects, tramiprosate, a prodrug of tramiprosate (e.g., ALZ-801), or an active metabolite of tramiprosate (e.g., 3-SPA) are administered orally.

As used herein, an amyloid plaque clearing agent refers to a chemical compound or biologic which clears insoluble amyloid plaques from the brain. Amyloid plaque clearing agents include immunotherapeutics (such as monoclonal antibodies, fusion proteins, soluble cytokine receptors, recombinant cytokines, small molecule mimetics, and cellular therapies) and polypeptides comprising bacteriophages. In some aspects, as part of a fifth embodiment, the amyloid plaque clearing agent is an antibody (e.g., Lecanemab, Aducanumab, Donanemab, Bapineuzumab, Solanezumab, Crenezumab, and Gantenerumab); a bacteriophage g3p-derived polypeptide (see e.g., WO 2013/082114, WO 2014/055515, WO 2014/193935, WO 2016/090022 and WO 2019/241628); or a vaccine directed against Abeta42 or Abeta 40 (e.g., AN-1792, CAD106, ACI-24, UB311, DNA-encoding Abeta 42 vaccines, and Lu AF20513). In some aspects, an amyloid plaque clearing agent is administered parenterally. In some aspects, an amyloid plaque clearing agent is administered intravenously.

In a sixth embodiment, the first period of time in the described administration protocols (e.g., in any one of the first to fifth embodiments) refers to an amount of time sufficient to reduce Aβ42 toxicity and/or to lower vascular amyloid burden. In other aspects, as part of a sixth embodiment, the first period of time in the described administration protocols (e.g., in any one of the first to fifth embodiments) refers to an amount of time sufficient to reduce the serum level of Aß40, Aß42, and/or p-tau₁₈₁ in the brain below a certain threshold level. In other aspects, as part of a sixth embodiment, the first period of time in the described administration protocols (e.g., in any one of the first to fifth embodiments) refers to an amount of time sufficient to reduce the serum level of Aß40 in the brain below a certain threshold level. In other aspects, as part of a sixth embodiment, the first period of time in the described administration protocols (e.g., in any one of the first to fifth embodiments) refers to an amount of time sufficient to reduce the serum level of Aß42 in the brain below a certain threshold level. In other aspects, as part of a sixth embodiment, the first period of time in the described administration protocols (e.g., in any one of the first to fifth embodiments) refers to an amount of time sufficient to reduce the serum level of a p-tau biomarker (e.g., p-tau₁₈₁) in the brain below a certain threshold level. In some aspects, as part of a sixth embodiment, the first period of time in the described administration protocols (e.g., in any one of the first to fifth embodiments) is at least about 6 months, at least about 7 months, at least about 8 months, at least about 9 months, at least about 10 months, at least about 11 months, at least about 12 months, at least about 18 months, or at least about 24 months. In some aspects, as part of a sixth embodiment, the first period of time in the described administration protocols (e.g., in any one of the first to fifth embodiments) is from about 6 months to about 24 months, about 6 months to about 18 months, about 6 months to about 12 months, about 10 months to about 14 months, or about 11 months to about 13 months. In some aspects, as part of a sixth embodiment, the first period of time in the described administration protocols (e.g., in any one of the first to fifth embodiments) is at least about 12 months. In some aspects, as part of a sixth embodiment, the first period of time in the described administration protocols (e.g., in any one of the first to fifth embodiments) is about 12 months.

In a seventh embodiment, the threshold level referred to for Aß40 and/or Aß42 in the sixth embodiment is characterized as a brain serum level of Aß40 and/or Aß42 that is at least 5% lower, at least 10% lower, at least 15% lower, at least 20% lower, at least 25% lower, at least 30% lower, at least 35% lower, at least 40% lower, at least 45% lower, at least 50% lower, at least 55% lower, at least 60% lower, at least 65% lower, at least 70% lower, or at least 75% lower than the brain serum level of Aß40 and/or Aß42 determined less than 30 days, less than 60 days, less than 90 days, less than 4 months, less than 5 months, less than 6 months, less than 7 months, less than 8 months, less than 9 months, less than 10 months, less than 11 months, or less than 12 months days prior to initiation of treatment. In one aspect, as part of a seventh embodiment, the threshold level referred to for Aß40 and/or Aß42 in the sixth embodiment is characterized as a brain serum level of Aß40 and/or Aß42 that is at least 5% lower than the brain serum level of Aß40 and/or Aß42 determined less than 60 days prior to initiation of treatment.

In an eighth embodiment, the threshold level referred to for a p-tau biomarker in the sixth embodiment is characterized as a brain serum level of p-tau₁₈₁ that is at least 30% lower, at least 35% lower, at least 40% lower, at least 45% lower, at least 50% lower, at least 55% lower, at least 60% lower, at least 65% lower, at least 70% lower, or at least 75% lower than the brain serum level of p-tau₁₈₁ determined less than 30 days, less than 60 days, less than 90 days, less than 4 months, less than 5 months, less than 6 months, less than 7 months, less than 8 months, less than 9 months, less than 10 months, less than 11 months, or less than 12 months days prior to initiation of treatment. In one aspect, as part of an eighth embodiment, the threshold level referred to for a p-tau biomarker is in the sixth embodiment is characterized as a brain serum level of such p-tau biomarker that is at least 40% lower than the brain serum level of that same p-tau biomarker determined less than 60 days prior to initiation of treatment. In some embodiments, the p-tau biomarker is p-tau₁₈₁. In some embodiments, the p-tau biomarker is p-tau₂₁₇. In some embodiments, the p-tau biomarker is p-tau₂₃₁. In some embodiments, the p-tau biomarker is p-tau₂₄₃.

In a ninth embodiment, the second period of time in the described administration protocols (e.g., in any one of the first to eighth embodiments) refers to an amount of time sufficient to clear amyloid plaque. In one aspect, as part of a ninth embodiment, the second period of time in the described administration protocols (e.g., in any one of the first to eighth embodiments) refers to an amount of time sufficient to clear pre-existing amyloid plaque, i.e., plaque that was present prior to the initiation of treatment. In one aspect, as part of a ninth embodiment, the second period of time in the described administration protocols (e.g., in any one of the first to eighth embodiments) refers to an amount of time sufficient to clear residual amyloid plaque, i.e., plaque that is present prior to administration of the amyloid plaque clearing agent. In one aspect, as part of a ninth embodiment, the second period of time in the described administration protocols (e.g., in any one of the first to eighth embodiments) is from about 3 months to about 12 months, from about 3 months to about 6 months, from about 6 months to about 18 months, or from about 6 months to about 12 months. In one aspect, as part of a ninth embodiment, the second period of time in the described administration protocols (e.g., in any one of the first to eighth embodiments) is from about 6 months to about 12 months. In one aspect, as part of a ninth embodiment, the second period of time in the described administration protocols (e.g., in any one of the first to eighth embodiments) ends when the subject has a negative amyloid PET scan.

In a tenth embodiment, the amyloid plaque clearing agent used in the described administration protocols (e.g., in any one of the first to ninth embodiments) is Lecanemab, Donanemab or Aducanumab. Alternatively, as part of a tenth embodiment, the amyloid plaque clearing agent used in the described administration protocols (e.g., in any one of the first to ninth embodiments) is Lecanemab or Aducanumab, wherein the effective amount of Donanemab and Aducanumab is about 10 mg/kg monthly and of Lecanemab is 10 mg/kg twice per month.

In an eleventh embodiment, the amyloid plaque clearing agent used in the described administration protocols (e.g., in any one of the first to tenth embodiments) is administered at a sub-therapeutic dose, e.g., less than 10 mg/kg/month for the amyloid plaque clearing agents Donanemab and Aducanumab, and less than 20 mg/kg/month for lecanemab.

In a twelfth embodiment, subjects are only treated by the present methods (including any one of the first to eleventh embodiments) if they have mild to moderate AD, which includes subjects with MMSE between 16-26. Alternatively, as part of a twelfth embodiment, subjects are only treated by the present methods (including any one of the first to eleventh embodiments) if they have moderate to severe AD, which includes subjects with MMSE<20 or subjects<22. In another alternative, as part of a twelfth embodiment, subjects are only treated by the present methods (including any one of the first to eleventh embodiments) if they have an MMSE score of ≥22. In another alternative, as part of a twelfth embodiment, subjects are only treated by the present methods (including any one of the first to eleventh embodiments) if an MMSE score of 22 to 28. In another alternative, as part of a twelfth embodiment, subjects are only treated by the present methods (including any one of the first to eleventh embodiments) if they have an MMSE score of 22 to 26. In another alternative, as part of a twelfth embodiment, subjects are only treated by the present methods (including any one of the first to eleventh embodiments) if they have an MMSE score of ≤21.

In a thirteenth embodiment, subjects are only to be treated by the present methods (including any one of the first to twelfth embodiments) if they meet certain parameters. In some aspects of this thirteenth embodiment, only subjects who: (a) have at least one APOE4 allele, i.e., APOE4 positive or APOE4⁺ subjects; or (b) who are APOE4-negative, i.e., APOE4− (non-carriers of the APOE4 allele), and have evidence of cerebral amyloid angiopathy on imaging; or (c) who are APOE4− and are being administered an anticoagulant therapy are treated. In some aspects, as part of a thirteenth embodiment, subjects are only to be treated by the present methods (including any one of the first to twelfth embodiments) if they are: (a) APOE4 homozygous, i.e. APOE4/4; or (b) who are APOE4− or APOE4 heterozygous (having only one APOE4 allele) and have evidence of cerebral amyloid angiopathy on imaging; or (c) who are APOE4− or APOE4 heterozygous and are being administered an anticoagulant therapy. In alternate aspects of the thirteenth embodiment, only subjects who are APOE4-positive are treated. In other aspects, only subjects who are APOE4 homozygous are treated. In still other aspects, only subjects who are APOE4 positive and either: (i) have evidence of cerebral amyloid angiopathy, or (ii) are being administered an anticoagulant therapy are treated. In yet other aspects, only subjects who are APOE4 homozygous and either: (i) have evidence of cerebral amyloid angiopathy, or (ii) are being administered an anticoagulant therapy are treated.

In a fourteenth embodiment, the effective dose of ALZ-801 used in the present methods (including any one of the first, second, and sixth to thirteenth embodiments) is about 530 mg/day. Alternatively, as part of a fourteenth embodiment, the effective dose of ALZ-801 used in the present methods (including any one of the first, second, and sixth to thirteenth embodiments) is about 265 mg BID.

In a fifteenth embodiment, ALZ-801 as defined herein (including any one of the first, second, and sixth to fourteenth embodiments) is administered to the subject orally.

In a sixteenth embodiment, ALZ-801 as defined herein (including any one of the first, second, and sixth to fifteenth embodiments) is formulated into a tablet, a capsule, a liquid, an orally dissolving tablet, a sachet, or sprinkles. In some aspects of a sixteenth embodiment, ALZ-801 as defined herein (including any one of the first, second, and sixth to fifteenth embodiments) is formulated into a capsule. In some aspects of a sixteenth embodiment, ALZ-801 as defined herein (including any one of the first, second, and sixth to fifteenth embodiments) is formulated into an instant release capsule. In other aspects of a sixteenth embodiment, ALZ-801 as defined herein (including any one of the first, second, and sixth to fifteenth embodiments) is formulated into an extended release capsule. In still other aspects of a sixteenth embodiment, each capsule comprises about 265 mg of ALZ-801.

### EXEMPLIFICATION

Evaluation of disease modifying effects from phase 2 trial of ALZ-801 (valiltramiprosate) in subjects with early Alzheimer's disease were conducted. The study enrolled 84 patients, with early AD, who carry the APOE4/4 or APOE3/4 genotype and have an MMSE score ranging from 22 to 30 (CDR-G 0.5 or 1). Each patient received 265 mg ALZ-801 twice daily. The average MMSE score of the subjects was 26 (mean age 69 years, 52% female). Subjects were either amyloid PET positive or A+/T+ on CSF assays. CSF criteria were: Ratio of Aβ42/40×10<0.61, and p-tau₁₈₁>61 pg/ml. CSF was evaluated at 52 weeks, and plasma biomarkers at every visit.

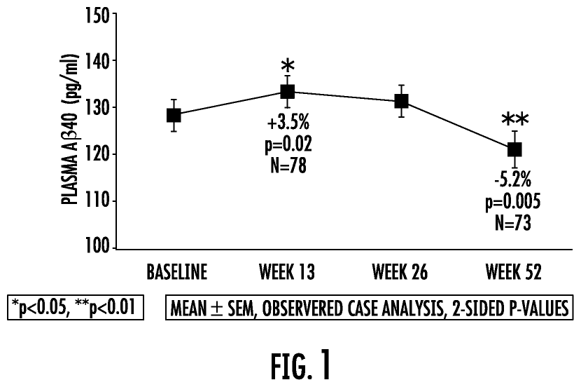

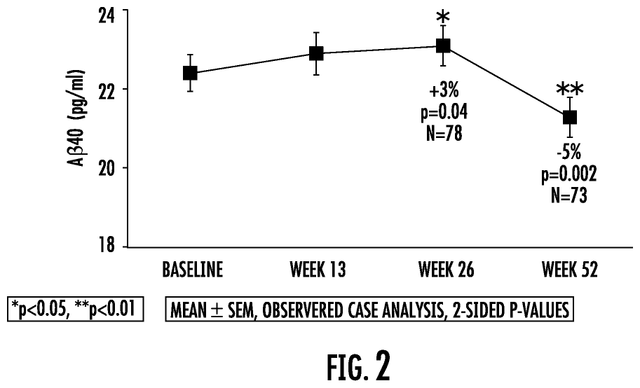

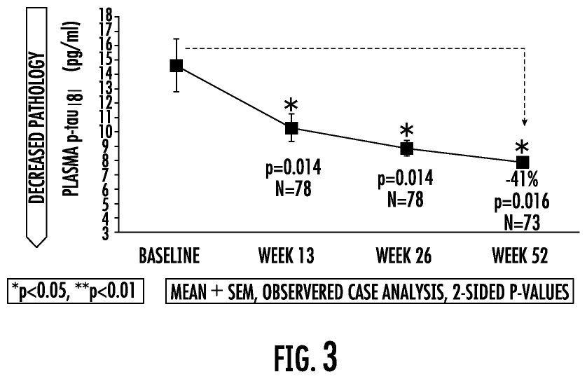

Subjects treated with ALZ-801 for showed significant effects on core plasma biomarkers of AD. Plasma levels of Aβ40 and Aβ42 both showed a biphasic response compared to baseline levels. See FIG. 1 and FIG. 2. Aβ40 showed significant early elevation at 13 weeks, followed by significant reduction of ~5% at 52 weeks. See FIG. 1. Aβ42 showed significant elevation at 26 weeks, followed by significant reduction of ~5% at 52 weeks. See FIG. 2. Plasma p-tau showed significant reduction starting at 13 weeks and progressed to 41% reduction at 52 weeks. See FIG. 3. These effects were associated with benefits on a composite cognitive outcome that tests memory and learning. ALZ-801 showed a significant reduction of HV atrophy by ~19-23% % when compared to a matched external control group from the ADNI-1 observational study. The HV preservation effects correlate with cognitive benefits (Spearman's coefficient r=0.27, p=0.02).

Over 1 year of ALZ-801 treatment, subjects showed no events of vasogenic edema (ARIA-E or ARIA-H) or macrohemorrhages. Given the safety of this drug and its ability to reduce Aβ40, leading to a postulated improvement in the overall health and function of cortical small and medium vessels, the following dosage protocol is contemplated as a safe and effective means for removing amyloid plaque in a subject having AD:
Phase 1: Administer 265 mg BID ALZ-801 for at least 1 year;
Phase 2: Administer an effective amount of an amyloid-plaque clearing agent with 265 mg BID ALZ-801 over a period of about 6 to 12 months period, or until patients achieve negative amyloid PET scan; and
Phase 3: Cease administration of the amyloid-plaque clearing agent and continue to administer 265 mg BID ALZ-801 thereafter.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

The contents of all references (including literature references, issued patents, published patent applications, and co-pending patent applications) that may be cited throughout this application are hereby expressly incorporated herein in their entireties by reference. Unless otherwise defined, all technical and scientific terms used herein are accorded the meaning commonly known to one with ordinary skill in the art.

1. A method of treating a subject suffering from Alzheimer's disease comprising the steps of:
a. administering to the subject an effective dose of tramiprosate, a tramiprosate prodrug, an active tramiprosate metabolite, or a deuterated form of any of the foregoing for a first period of time;
b. co-administering to the subject for a second period of time:
i. an effective dose of tramiprosate, a tramiprosate prodrug, an active tramiprosate metabolite, or a deuterated form of any of the foregoing; and
ii. an effective dose of an amyloid plaque clearing agent; and
c. administering to the subject an effective dose of tramiprosate, a tramiprosate prodrug, an active tramiprosate metabolite, or a deuterated form of any of the foregoing thereafter.

2. The method of claim 1, wherein in each of steps a., b., and c., the subject is administered a tramiprosate prodrug that is a tramiprosate amino acid prodrug.

3. The method of claim 2, wherein the tramiprosate amino acid prodrug has the chemical formula:

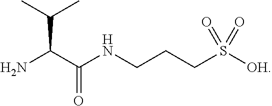

4. The method of claim 1, wherein in each of steps a., b., and c., the subject is administered tramiprosate.

5. The method of claim 1, wherein in each of steps a., b., and c., the subject is administered a tramiprosate metabolite that has the chemical formula:

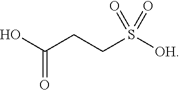

6. The method of any one of claims 1-5, wherein the amyloid plaque clearing agent is an antibody or a bacteriophage g3p-derived polypeptide.

7. The method of claim 6, wherein the amyloid plaque clearing agent is selected from Lecanemab, Aducanumab, Bapineuzumab, Solanezumab, Crenezumab, and Gantenerumab.

8. The method of claim 6, wherein the amyloid plaque clearing agent is Lecanemab or Aducanumab.

9. The method of any one of claims 1-8, wherein the first period of time is at least about 12 months.

10. The method of any one of claims 1-9, wherein the first period of time ends when a serum biomarker selected from Aß40, Aß42, and p-tau drops below a threshold level.

11. The method of claim 9, wherein the p-tau serium biomarker is p-tau₁₈₁.

12. The method of claim 10, wherein the threshold level for Aß40 or Aß42 is at least 5% lower than the level determined less than sixty days prior to initiation of treatment.

13. The method of claim 10 or 11, wherein the threshold level for p-tau or p-tau₁₈₁ is at least 40% lower than the level determined less than sixty days prior to initiation of treatment.

14. The method of any one of claims 1-13, wherein the second period of time is from about 6 months to about 12 months.

15. The method of any one of claims 1-13, wherein the second period of time ends when the subject has a negative amyloid PET scan.

16. The method of claim 14 or 15, wherein the amyloid plaque clearing agent is administered at a sub-therapeutic dose.

17. The method of any one of claims 1-16, wherein the subject is undergoing treatment with an anticoagulant.

18. The method of any one of claims 1-17, wherein the subject has evidence of cerebral amyloid angiopathy (“CAA”).

19. The method of any one of claims 1-18, wherein the subject is treated only if the subject is ApoE4⁺.

20. The method of claim 19, wherein the subject is treated only if the subject is ApoE4 homozygous.

21. The method of any one of claims 1-20, wherein the subject is treated only if the subject has an MMSE score of ≥22.

22. The method of any one of claims 1-21, wherein the effective dose of the tramiprosate amino acid prodrug of the chemical formula: 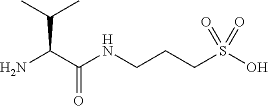
is about 265 mg BID.
